(19) United States
(12) Patent Application Publication
(10) Pub. No.: US 2018/0133411 A1
(43) Pub. Date: May 17, 2018

(54) Systems and Methods for Detecting and Visualizing Blood Vessels

(71) Applicant: Xiang Yu, Vienna, VA (US)

(72) Inventor: Xiang Yu, Vienna, VA (US)

(21) Appl. No.: 15/351,398

(22) Filed: Nov. 14, 2016

(51) Int. Cl. A61M 5/42; A61B 5/1455

(52) U.S. Cl. CPC A61M 5/427; A61B 5/14552

(57) ABSTRACT

The present invention relates to systems and methods for detecting and visualizing blood vessels. One of the implementations of a blood vessel detecting and visualizing system comprises a sensing device, a processing circuitry, and a visualization device. The sensing device detects a light signal from a blood vessel under the body surface and the blood vessel's surrounding tissues. The processing circuitry differentiates the signal from the blood vessel and the signal from the surrounding tissues and obtains an image of the blood vessel. The visualization device provides a visualization of the blood vessel on the body surface.

### TECHNICAL FIELD

The present invention generally relates to detection and visualization of blood vessels. More particularly, the present invention relates to systems and methods for detecting a light signal from a blood vessel under the body surface and creating a visualization of the blood vessel on the body surface. Providing such visualization of the blood vessel facilitates access to the blood vessel in a variety of medical procedures and treatments.

### BACKGROUND OF THE INVENTION

Accessing a blood vessel is a critical step in many medical treatments. In practice, getting access has proven difficult in many circumstances, especially when the blood vessel to be accessed is located deep under the body surface such as an artery.

Some solutions have been proposed to create a visualization of veins that are just under the skin for easier access. However, many procedures such as arterial blood gas (ABG), arterial catheterization, central venous catheterization, etc., require quick and efficient access to blood vessels embedded deeper under the skin such as the radial artery, superficial femoral artery, a large vein in the neck (internal jugular vein or external jugular vein), chest (subclavian vein) or groin (femoral vein). Medical professionals may perform such procedures based on touch-and-feel or through the aid of an ultrasound. The former often requires multiple attempts even for highly experienced practitioners, leading to unnecessary pain and suffering for the patients. The latter requires expensive and usually bulky equipment and the visualization is on a screen away from the patient rather than directly on the patient's body surface, which distracts the practitioner.

Accordingly, there remains a need for a solution that enables fast and accurate access to blood vessels located deep underneath the skin.

### SUMMARY

The present invention describes systems and methods for detecting a blood vessel under the body surface and creating a visualization of the blood vessel on the body surface. In one embodiment, the blood vessel detecting and visualizing system comprises a detecting device and a visualization device. The detecting device further comprises a sensor and a processing circuitry. The sensor detects a light signal from the blood vessel and a signal from the blood vessel's surrounding tissues. The processing circuitry is configured to differentiate the light signal from the blood vessel from the light signal from the blood vessel's surrounding tissues and obtain an image of the blood vessel. The visualization device is separate from the detecting device and can be attached to the body surface. The visualization device is configured to create a visualization of the blood vessel on the body surface. Upon detection of the blood vessel, the detecting device triggers a marking mechanism and creates a visible mark of the blood vessel on the visualization device attached to the body surface. Through visualization of a blood vessel, the system facilitates access to the blood vessel in a variety of medical procedures or treatments.

### BRIEF DESCRIPTION OF THE DRAWING

FIG. 1 illustrates an embodiment of the photoptic blood vessel detecting and visualizing system.

FIG. 2 illustrates the optical parts and the processing circuitry of the photoptic blood vessel detecting device.

FIG. 3 schematically shows the assembly of all the components in the photoptic blood vessel detecting device.

FIGS. 4A and 4B each illustrates an embodiment of the blood vessel visualization device.

### DETAILED DESCRIPTION

The blood transports oxygen through hemoglobin molecules found on red blood cells. A hemoglobin molecule consists of two parts namely the globin portion and a heme group. The globin portion is a protein made up of four highly folded polypeptide chains. The heme group is a non-protein group containing four iron molecules. Each of the four iron molecules combine irreversibly with an oxygen molecule. Because of the presence of iron, hemoglobin appears reddish in color when combined with oxygen and appears bluish when deoxygenated. Deoxygenated blood absorbs more red light than oxygenated blood. Hence, a fully oxygenated arterial blood is red in color while deoxygenated venous blood is bluish.

The present invention disclosed herein solves the problem by detecting and creating a direct visualization of the target blood vessel on the body surface, which facilitates a medical practitioner in obtaining easier and faster access to the blood vessel. While some prior arts have the capacity of detecting and visualizing veins that are close to the skin, the current invention is sensitive to detect blood vessels that are embedded deeper in the body such as the radial artery. Further, the present invention decouples the blood vessel detecting device from the blood vessel visualization device and retains the visualization of the blood vessel even after the detecting device is removed from the body, thereby providing easy, unobstructed access to the blood vessel after it is detected.

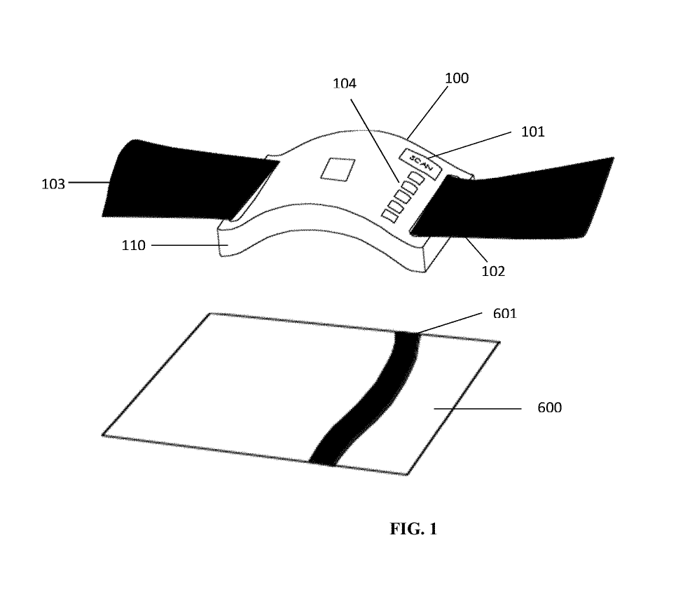

Referring to FIG. 1, according to one embodiment, the blood vessel detecting and visualizing system comprises two parts: the blood vessel detecting device 100 and the blood vessel visualization device 105. The blood vessel detecting device 100 includes a housing 110 which has a hard casing top secured by a pair of straps 102 and 103 and a button 101 that initiates the scanning to detect and map an artery. Once the location of the artery is mapped, the blood vessel detecting device 100 triggers the visualization mechanism of the visualization device 600 and creates a mark of the artery 601 on the visualization device 600. The blood vessel detecting device 100 allows a user to see when the device is in use and analyzing a blood vessel, as well as the battery life through the use of one or more indicators 104, such as light emitting diodes (LEDs), on the housing 110.

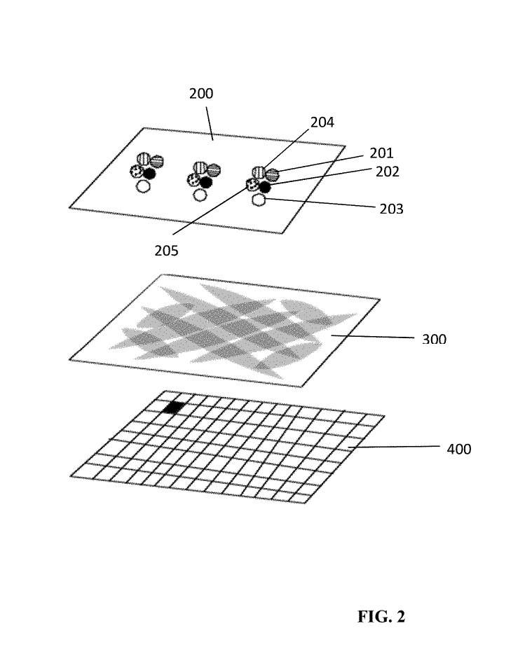

Referring to FIG. 2, the blood vessel detecting device comprises visible 201, infrared 202, and ultraviolet 203 LEDs that are attached to a board 200 and are used to detect the blood vessel and mark the location of the blood vessel on the blood vessel visualization device. The blood vessel detecting device may further include a light diffuser 300 which helps reduce artifacts by refracting non-absorbed light, and/or an adaptive filter 400 which helps remove noise from the light signals.

Oximetry is the term used to refer to the optical measurement of the saturation of oxyhemoglobin in the blood. All oximetry techniques are based on spectrophotometry. Spectrophotometry is based on the principle that all atomic molecules vibrate in a unique pattern for each individual substance. As light from LEDs 201, 202, and 203 passes through a given substance such as a blood vessel containing blood cells, the frequency of light that is identical to the frequency of that substance is absorbed, but the other frequencies are reflected.

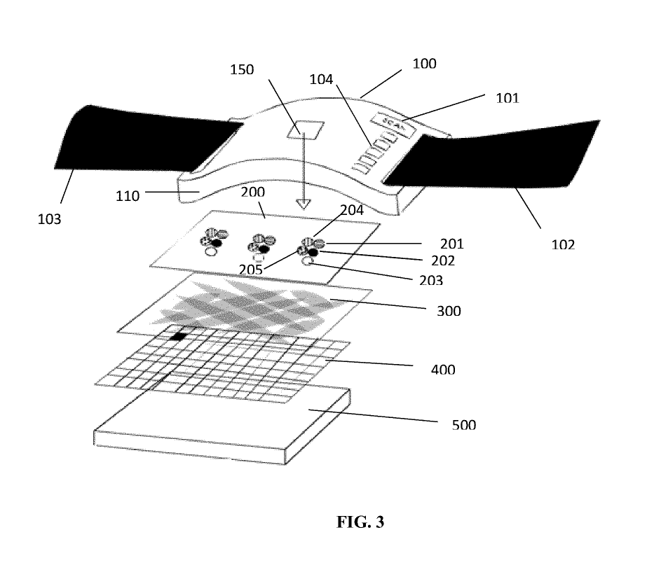

Referring to FIG. 3, the blood vessel detecting device 100 comprises a processing circuitry 150 for processing light signals. The optical components shown in FIG. 2, the processing circuitry 150, and all other components of the detecting device 100 are secured by a detachable bottom casing 500 that is part of the housing 110. The processing circuitry 150 includes spectrophotometers for measuring the amount of light that is not absorbed by the given substance. In general, the oximeter measures the relationship between the concentration of a solute to the amount of light transmitted through a solution based on the Beer-Lambert Law. The Beer-Lambert Law is based on the principle that the sum of the absorbed and transmitted light equals the incident light.

Oxyhemoglobin (HbO2) and deoxyhemoglobin (Hb) absorb light at certain wavelengths and have different light absorption characteristics. For instance, in traditional oximeters, two light emitting diodes emit light at 660 nm (red) and 940 nm (infrared) wavelengths. At these wavelengths both oxyhemoglobin and deoxyhemoglobin have different absorption spectra. When light from LEDs 201, 202, and 203 is passed through human tissue, the ratio of absorbances at these two wavelengths is then used to estimate the arterial blood oxygen saturation by the processing circuitry 150. The light that is not absorbed is reflected back. The photodiode detector 205 accepts the lights that are reflected back and the processing circuitry analyzes the different absorbance ratios of the lights emitted from the LEDs 201, 202, and 203.

In addition, the pulsatile signal generated by the artery, which is relatively independent of non-pulsatile arterial blood, venous and capillary blood, and other tissues, can be adopted to distinguish the absorbance of arterial blood and other absorbers such as skin, soft tissue, venous and capillary blood by analyzing light from another LED 204. The absorbance of arterial blood pulsations are similar to an “alternating current” or simply an AC component while the absorbance from other tissue are always a constant termed as an DC component, which is similar to a “direct current.” The AC component pulsations are caused by the systolic volume expansion of the arteriolar bed, which causes the increase in optical path length thus increasing the absorbance. Pulse oximetry is based on the assumption that the arterial blood is the only source of pulsatile absorbance while any other fluctuating light absorbers will act as a source of error. The pulsatile signal is sensitive to motion artifacts, and a third wavelength from LED 204 may be used to reduce such artifacts. The blood vessel detecting device may include a light diffuser 300 which helps reduce artifacts by refracting non-absorbed light. The third wavelength 204 is one at which oxyhemoglobin and deoxyhemoglobin have substantially the same absorption or so called isosbestic point. The light absorbances of the two types of hemoglobins can be calibrated by the processing circuitry 150 to reduce interferences caused by other artifacts.

The blood vessel detecting device 100 utilizes the differences in light absorbances between a blood vessel and its surrounding tissues to locate the blood vessel. Both an infrared light and a visible light are emitted from the infrared LED 202 and the visible LED 201 on device, respectively, through the tissue under a body surface. At the same time, a third light may be emitted from the ultraviolet LED 204 on the device through the tissue to that serves as a reference to reduce the background noise from the patient's body movement or other artifacts, thereby further enhancing the distinctions between the blood vessel and its surrounding tissue.

In preferred embodiments, the blood vessel detecting device further includes an adaptive filter 400 as shown in FIGS. 2 and 3 to remove noise from the light signals that it is measuring. Adaptive filters require a reference noise that is correlated to the spectrum of noise found in the composite signal. An adaptive filter comprises a digital filter which processes the light signals. It also comprises an adaptive algorithm that adjusts the coefficients of that filter to prevent non-light signals from being processed. The filter is made up of a matrix of cells that are substantially smaller than the diameter of the blood vessel. This allows the sensor to accurately detect the location of the blood vessel.

The adaptive filter 400 allows an infrared light and a visible light emitted respectively from the infrared LED 202 and visible LED 201 to pass through each cell while the processing circuitry 150 measures and records the ratio of the two lights reflected or transmitted from the tissue underneath that cell. When measurements from all the cells have been obtained, the processing circuitry 150 calculates the differences between light absorbance across all the cells and creates a density map of the light absorbance. The adaptive filter 400 also prevents other background noise from reaching the sensor based on the different frequencies to enhance the sensitivity of the density map. The density map can be correlated to the level of blood oxygen under the surface of the body, which is further correlated with the location of the blood vessel. The processing circuitry 150 thus can create an image of the location of blood vessels based on this density map.

In one embodiment of the blood vessel detecting and visualizing device, the adaptive filter 400 is composed of a matrix of liquid crystal (LC) particles. The LC particles respond to an electric field controlled by the processing circuitry and change their phases in the presence or absence of that electric field. In a first phase, the LC particles are oriented in such a way to allow light to pass through. In a second phase, the LC particles are oriented in such a way to block the light. Hence, by controlling the electric field corresponding to different positions of the LC matrix, only selected light can pass through the LC particles at specific positions at a selected time.

Once the image of blood vessels is created, the processing circuitry 150 then opens only those cells of the adaptive filter 400 above the location of the blood vessel allowing light from the LEDs 201, 202, 203 to pass through. The processing circuitry 150 then emits a light through the open cells onto the visualization device 600 which contains a light sensitive ink and thus creates a real visual mark 601 of the blood vessel on the body surface. The real visual mark 601 then aids a medical practitioner to perform the necessary procedure to access that blood vessel.

In alternative embodiments, the visualization device 600 may contain heat sensitive ink and the processing circuitry 150 includes a heating element which applies an adequate amount of heat and causes the heat sensitive ink contained in the visualization device 600 to change color and creates a visualization of the blood vessel.

In other embodiments, instead of using an adaptive filter, the blood vessel detecting device 100 may divide its surface into grids that are substantially smaller than the diameter of the blood vessel and the processing circuitry 150 stores the location of each grid such that the density map of light absorbance can be stored using the grids.

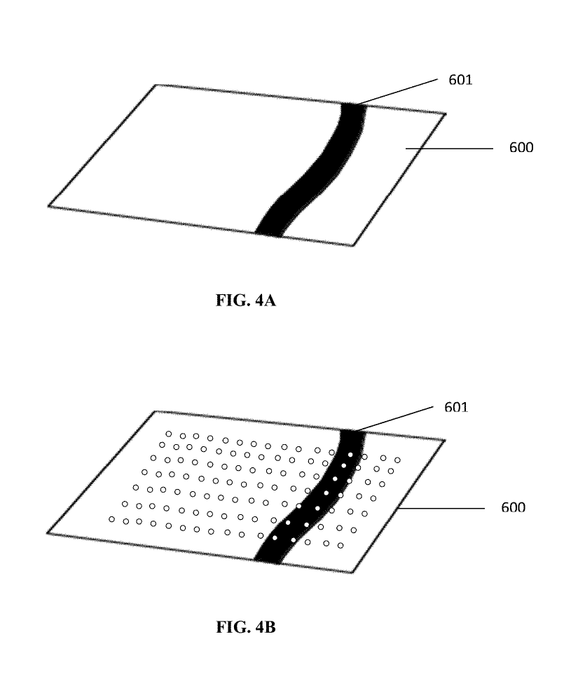

FIGS. 4A and 4B each illustrates an embodiment of the visualization device. In FIG. 4A, the visualization device is a patch that can be applied to the body surface. The patch contains light sensitive ink that can react to the light emitted from the blood vessel detecting device and provide a visualization indicating the location and shape of the blood vessel on the patch.

FIG. 4B provides another embodiment of the visualization device similar to the patch illustrated in FIG. 4A, except that the patch is perforated. The perforation provides easy and safe access to the blood vessel as a medical practitioner can easily thread a need through the holes on the patch.

In preferred embodiments, the visualization devices are sterile, disposable patches that can be safely and conveniently used in medical treatments.

Other features, advantages, and implementations of the present disclosures, not expressly disclosed herein, will be apparent to one of ordinary skills in the art upon examination of the following detailed description and accompanying drawings. It is intended that such implied implementations of the present disclosure be included herein.

What is claimed is:

1. A system for detecting and marking a blood vessel, comprising:
a. a visualization device; and
b. a detecting device comprising:
i. a sensor that detects a first signal from said blood vessel and a second signal from said blood vessel's surrounding tissues; and
ii. a processing circuitry that differentiates said blood vessel from its surrounding tissues upon said first signal and said second signal and creates a visible mark of said blood vessel on said visualization device.

2. The system in claim 1, wherein said blood vessel is an artery.

3. The system in claim 1, wherein said blood vessel is a vein.

4. The system in claim 1, wherein said sensor detects a photoptic signal from said blood vessel.

5. The system in claim 4, wherein said photoptic signal is correlated with the oxygen level of the blood.

6. The system in claim 1, wherein said sensor detects a piezoelectric signal from said blood vessel.

7. The system in claim 6, wherein said piezoelectric signal is correlated with the pressure of an arterial wall.

8. The system in claim 1, wherein said visualization device is a patch that can adhere to the body surface and display said visible mark.

9. The system in claim 9, wherein said patch is disposable.

10. The system in claim 9, wherein said patch displays said mark upon application of a light signal transmitted by said processing circuitry indicating position of said blood vessel being detected.

11. The system in claim 10, wherein said light signal is ultraviolet.

12. The system in claim 8, wherein said patch is perforated.

13. A system for detecting and marking a blood vessel, comprising:
a. a visualization device; and
b. a detecting device comprising:
i. a sensor that detects a first signal from said blood vessel and a second signal from said blood vessel's surrounding tissues, said sensor comprising a pre-arrangement of sensory elements each corresponds to a specific position on a body surface when said sensor is applied to said body surface;
ii. a processing circuitry that differentiates said blood vessel from its surrounding tissues upon said first signal and said second signal and creates a visible mark of said blood vessel on said visualization device.

14. The system in claim 13, wherein said visualization component is a patch that can adhere to the body surface and display said visible mark.

15. The system in claim 14, wherein said patch is disposable.

16. The system in claim 14, wherein said patch is perforated.

17. A method for detecting and marking a blood vessel, comprising:
a. detecting a first signal from said blood vessel and a second signal from said blood vessel's surrounding tissues;
b. processing said first signal and said second signal to differentiate said blood vessel from its surrounding tissues; and
c. creating a visible mark of said blood vessel on a visualization device attached to the body surface.

18. The method in claim 17, wherein said visualization device is a disposable patch.

19. The method in claim 18, wherein said patch is perforated.
